(19) United States
(12) Patent Application Publication
(10) Pub. No.: US 2022/0339991 A1
(43) Pub. Date: Oct. 27, 2022

(54) AIR STERILIZATION DEVICE FOR VEHICULAR AIR CONDITIONER

(71) Applicants: Hyundai Motor Company, Seoul (KR); Kia Corporation, Seoul (KR)

(72) Inventors: Jae Seung Lee, Hwaseong-si (KR); Dong Ho Kwon, Yongin-si (KR)

(73) Assignees: Hyundai Motor Company, Seoul (KR); Kia Corporation, Seoul (KR)

(21) Appl. No.: 17/511,838

(22) Filed: Oct. 27, 2021

(30) Foreign Application Priority Data
Apr. 27, 2021 (KR) 10-2021-0054089

(51) Int. Cl. B60H 3/06; A61L 9/20; B60H 1/12

(52) U.S. Cl. CPC B60H 3/0608; A61L 9/205; B60H 1/12; A61L 2209/12; B60H 2003/0675; A61L 2209/16

(57) ABSTRACT

An air sterilization device for a vehicular air conditioner may include an air conditioner case, which includes an air inlet provided at an intake side of the air conditioner case and an air outlet provided at a discharge side of the air conditioner case and which includes an air path therein, a photocatalytic unit, which is configured to blow internal air or external air toward the air outlet of the air conditioner case and a surface of which is coated with a photocatalyst, and an optical source unit configured to radiate ultraviolet rays to the photocatalytic unit to thus cause a photocatalytic reaction at the photocatalytic unit, generating hydroxyl radicals.

### CROSS-REFERENCE TO RELATED APPLICATION

The present application claims priority to Korean Patent Application No. 10-2021-0054089 filed on Apr. 27, 2021, the entire contents of which is incorporated herein for all purposes by this reference.

### BACKGROUND OF THE INVENTION

### Field of the Invention

The present invention relates to an air sterilization device for a vehicular air conditioner. More particularly, it relates to an air sterilization device for a vehicular air conditioner, which is configured to function to sterilize bacteria and viruses that are not removed by a filter of the air conditioner and to supply the cleaned air to a passenger compartment.

### Description of Related Art

Generally, with the increasing realization that infection by airborne viruses in the air spreads through droplets in the air and that scattering of the droplets and generation of high concentrations of fine dust causes the spread of the virus, there is demand for a technology for eliminating bioaerosols in the air and viruses which are nanoparticle-sized microorganisms.

HEPA filters and electrostatic dust collection techniques may be considered representative technologies for efficiently collecting harmful microorganisms such as viruses in the air.

An air purifier, to which an antibacterial filter, which is manufactured in such a way as to coat a filter with an antibacterial material, is applied to kill harmful microorganisms, utilizes antibacterial technology for killing microorganisms adhering to the filter by generating ion clusters in front of the filter and causing the microorganisms to react with the ions, and a sterilization technology for killing microorganisms by passing the microorganisms through plasma.

However, because large particulate matter easily accumulates on the above-mentioned conventional antibacterial filter due to contamination, there is a problem in that it is impossible in practice to kill harmful nanoparticle-sized microorganisms.

Because, in an air purifier for eliminating fine dust using a HEPA filter, a clean air delivery rate (CADR) is lowered due to loss of a large of amount of fluid pressure caused by the HEPA filter, there is a problem in that it is impossible for the air purifier to efficiently and instantaneously sterilize air contaminated by the virus.

Because such a filter for air purification is configured to perform filtration only once on the contaminated air introduced into the air purifier, bacteria, viruses and the like, which are not removed by the filter, may be introduced into a passenger compartment, increasing the risk of spread of infectious diseases.

Furthermore, although plasma is known to be effective for extinction of bacteria and viruses in the air, there is a problem in that the reaction is ineffective because the plasma of active contact with plasma, within which bacteria and viruses are killed, is narrow, and radicals cannot be spread far by a fan.

The information disclosed in this Background of the Invention section is only for enhancement of understanding of the general background of the invention and may not be taken as an acknowledgement or any form of suggestion that this information forms the prior art already known to a person skilled in the art.

### BRIEF SUMMARY

Various aspects of the present invention are directed to providing an air sterilization device configured for a vehicular air conditioner including a photocatalytic unit, which is manufactured in such a way as to coat a blower fan with a photocatalyst, and optical sources configured to radiate ultraviolet rays to the photocatalytic unit to thus activate a photocatalytic reaction, generating hydroxyl radicals, wherein the photocatalytic unit and the optical sources are mounted in an air path, into which internal air or external air is introduced, to perform sterilization using ultraviolet rays and sterilization resulting from the oxidizing action of hydroxyl radicals generated by the ultraviolet rays, making it possible to fulfill efficient sterilization of contaminants.

Various aspects of the present invention are directed to providing an air sterilization device configured for a vehicular air conditioner including an air conditioner case, which includes an air inlet provided at an intake side of the air conditioner case and an air outlet provided at a discharge side of the air conditioner case and which includes an air path therein, a photocatalytic unit, which is configured to blow internal air or external air toward the air outlet of the air conditioner case and a surface of which is coated with a photocatalyst, and an optical source unit configured to radiate ultraviolet rays to the photocatalytic unit to thus cause a photocatalytic reaction at the photocatalytic unit, generating hydroxyl radicals.

In various exemplary embodiments of the present invention, the optical source unit may include a first optical source, mounted on a coupling mount provided at a center portion of the photocatalytic unit to radiate ultraviolet rays to an internal surface of the photocatalytic unit, and a second optical source, configured to radiate ultraviolet rays to an external surface of the photocatalytic unit.

In another exemplary embodiment of the present invention, the first optical source may include a plurality of first optical sources, which are disposed on an external circumferential surface of the coupling mount positioned at the center portion of the photocatalytic unit at angular intervals of 90 degrees and which are oriented to be inclined downwards and to face the internal surface of the photocatalytic unit.

In yet another exemplary embodiment of the present invention, the second optical source may include a plurality of second optical sources, the number of which is the same as a number of the plurality of first optical sources and which are mounted on the air conditioner case to respectively face the first optical sources.

In yet another exemplary embodiment of the present invention, the second optical sources may be mounted at a position on the air conditioner case which is aligned with a line extending through the center portion of the photocatalytic unit.

In still yet another exemplary embodiment of the present invention, the second optical source may selectively include an optical lens coupled thereto when the second optical source is positioned at a distance from the external surface of the photocatalytic unit which is smaller than a predetermined distance.

In a further exemplary embodiment of the present invention, the photocatalytic unit may be a blower fan.

The methods and apparatuses of the present invention have other features and advantages which will be apparent from or are set forth in more detail in the accompanying drawings, which are incorporated herein, and the following Detailed Description, which together serve to explain certain principles of the present invention.

### BRIEF DESCRIPTION OF THE DRAWINGS

FIG. 1 is a view exemplarily illustrating an air conditioner case of an air sterilization device of a vehicular air conditioner according to various exemplary embodiments of the present invention;

FIG. 2 is a cross-sectional view taken along line A-A′ in FIG. 1, which illustrates the air sterilization device of the vehicular air conditioner according to the exemplary embodiment of the present invention;

FIG. 3 is an enlarged view of the square region in FIG. 2, which illustrates the sterilization mechanism of the air sterilization device of the vehicular air conditioner according to the exemplary embodiment of the present invention;

FIG. 4 is a cross-sectional view taken along line B-B′ in FIG. 1, which illustrates the position of an optical source unit of the air sterilization device of the vehicular air conditioner according to the exemplary embodiment of the present invention;

FIG. 5 is a view exemplarily illustrating the optical source unit of the air sterilization device of the vehicular air conditioner according to the exemplary embodiment of the present invention;

FIG. 6 is a view exemplarily illustrating a photocatalytic unit of the air sterilization device of the vehicular air conditioner according to the exemplary embodiment of the present invention;

FIG. 7 and FIG. 8 are views exemplarily illustrating the position of a second optical source of the air sterilization device of the vehicular air conditioner according to the exemplary embodiment of the present invention; and

FIG. 9 and FIG. 10 are views exemplarily illustrating an optical lens of the air sterilization device of the vehicular air conditioner according to the exemplary embodiment of the present invention.

It may be understood that the appended drawings are not necessarily to scale, presenting a somewhat simplified representation of various exemplary features illustrative of the basic principles of the present invention. The specific design features of the present invention as disclosed herein, including, for example, specific dimensions, orientations, locations, and shapes, will be determined in portion by the particular intended application and use environment.

In the figures, the reference numbers refer to the same or equivalent parts of the present invention throughout the several figures of the drawing.

### DETAILED DESCRIPTION

Reference will now be made in detail to various embodiments of the present invention(s), examples of which are illustrated in the accompanying drawings and described below. While the present invention(s) will be described in conjunction with exemplary embodiments of the present invention, it will be understood that the present description is not intended to limit the present invention(s) to those exemplary embodiments. On the other hand, the present invention(s) is/are intended to cover not only the exemplary embodiments of the present invention, but also various alternatives, modifications, equivalents and other embodiments, which may be included within the spirit and scope of the present invention as defined by the appended claims.

Hereinafter, exemplary embodiments of the present invention will be described in detail with reference to the accompanying drawings.

The objects described above, as well as other objects, features and advantages, will be clearly understood from the following exemplary embodiments with reference to the appended drawings.

However, the present invention is not limited to the embodiments, and will be embodied in different forms. The exemplary embodiments are suggested only to offer a thorough and complete understanding of the included contents and to sufficiently inform those skilled in the art of the technical concept of the present invention. The present invention is defined only by the categories of the claims.

Furthermore, in the following description of embodiments included herein, if it is determined that a detailed description of known functions or configurations related to the present invention would make the subject matter of the present invention unclear, such detailed description is omitted.

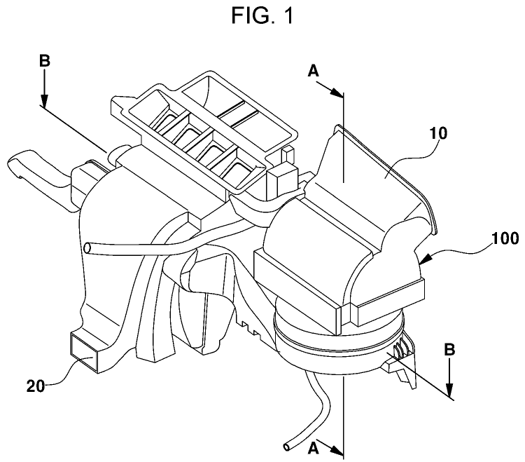

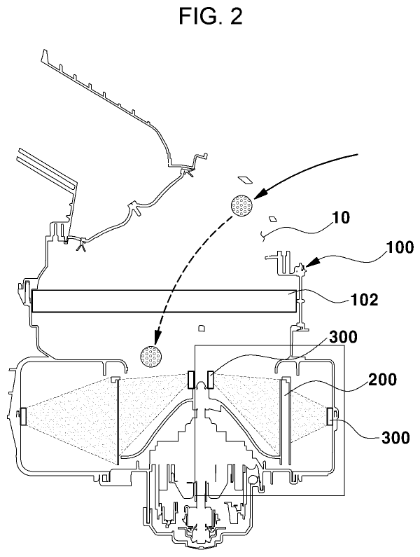

FIG. 1 is a view exemplarily illustrating an air conditioner case of an air sterilization device of a vehicular air conditioner according to various exemplary embodiments of the present invention. FIG. 2 is a cross-sectional view taken along line A-A′ in FIG. 1, which illustrates the air sterilization device of the vehicular air conditioner according to the exemplary embodiment of the present invention.

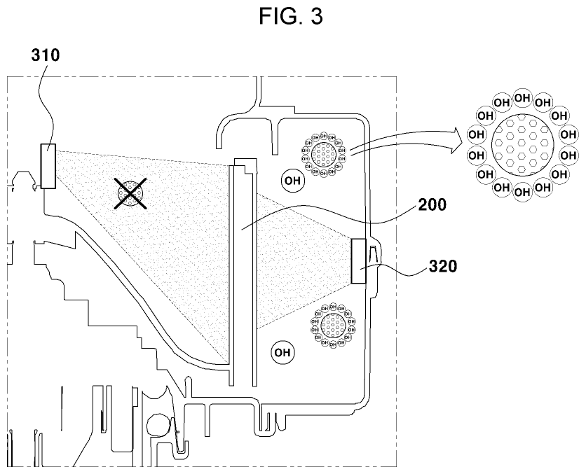

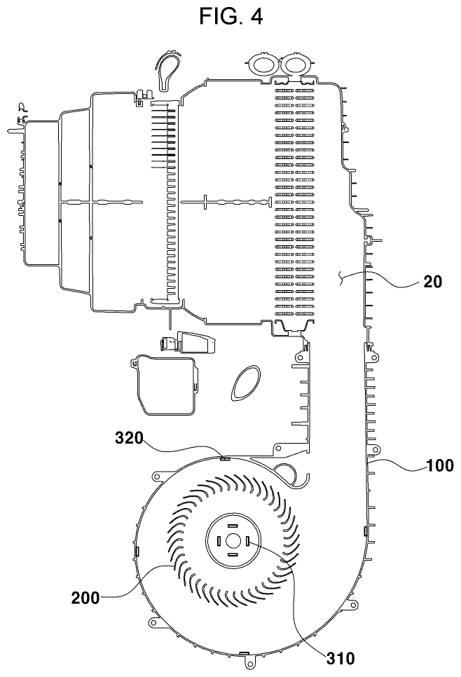

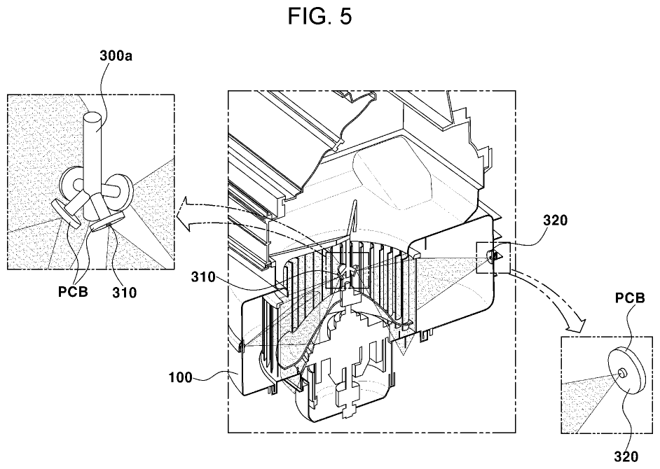

FIG. 3 is an enlarged view of the square region in FIG. 2, which illustrates the sterilization mechanism of the air sterilization device of the vehicular air conditioner according to the exemplary embodiment of the present invention. FIG. 4 is a cross-sectional view taken along line B-B′ in FIG. 1, which illustrates the position of an optical source unit of the air sterilization device of the vehicular air conditioner according to the exemplary embodiment of the present invention. FIG. 5 is a view exemplarily illustrating the optical source unit of the air sterilization device of the vehicular air conditioner according to the exemplary embodiment of the present invention.

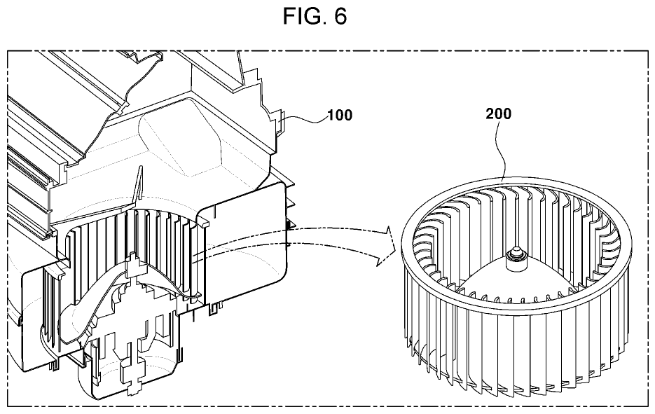

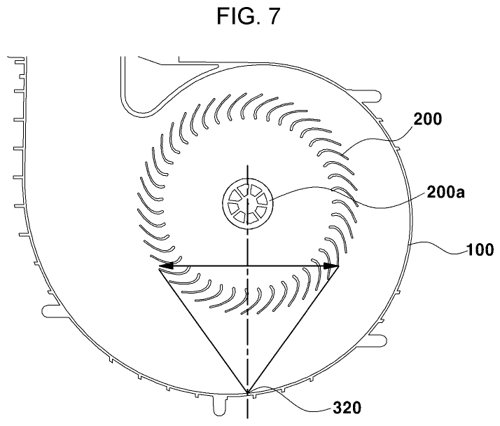

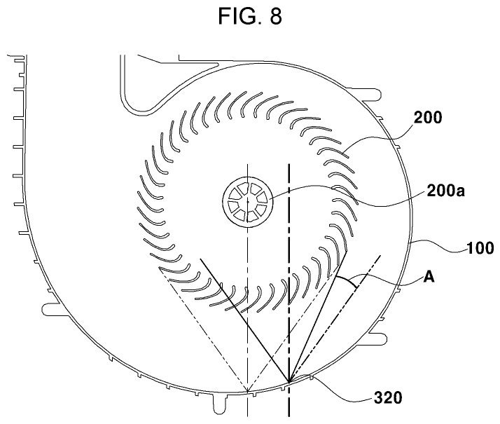

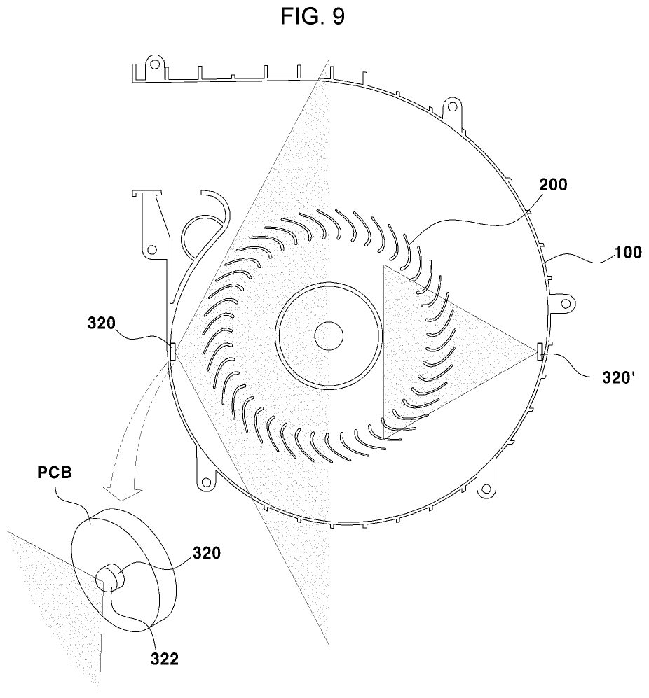

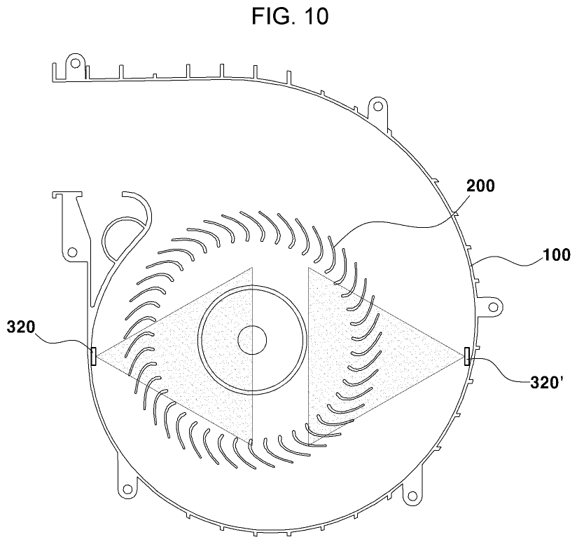

FIG. 6 is a view exemplarily illustrating a photocatalytic unit of the air sterilization device of the vehicular air conditioner according to the exemplary embodiment of the present invention. FIG. 7 and FIG. 8 are views exemplarily illustrating the position of a second optical source of the air sterilization device of the vehicular air conditioner according to the exemplary embodiment of the present invention. FIG. 9 and FIG. 10 are views exemplarily illustrating an optical lens of the air sterilization device of the vehicular air conditioner according to the exemplary embodiment of the present invention.

As illustrated in FIG. 1 and FIG. 2, the air sterilization device of the vehicular air conditioner according to the exemplary embodiment of the present invention includes an air conditioner case 100, a photocatalytic unit 200 and an optical unit 300.

The air conditioner case 100 is provided at the intake thereof with an air inlet 10 and at the discharge side thereof with an air outlet 20, and is provided therein with an air path including an air-conditioning filter 102.

The photocatalytic unit 200 is provided in the air sterilization device to forcibly blow internal air or external air toward the air outlet 20 of the air conditioner case 100.

As illustrated in FIG. 6, the photocatalytic unit 200 may be a blower fan, which operates to move and purify the internal air or the external air having passed the air-conditioning filter 102, and then discharge the same through the air outlet 20.

The surface of the photocatalytic unit 200 is coated with a photocatalyst.

As illustrated in FIG. 3, the photocatalytic unit 200 is configured to cause a photocatalytic reaction by radiation of ultraviolet (UV) rays, that is, ultraviolet-C (UVC) rays from the optical source unit 300 to thus generate hydroxyl (OH−) radicals.

The photocatalytic unit 200 causes a photocatalytic reaction due to the ultraviolet rays radiated from the optical source unit 300. By the oxidizing action of the hydroxyl radicals generated by the photocatalytic reaction, pathogens introduced into the air conditioner case 100, that is, bacteria, viruses and the like, are killed.

When the photocatalytic unit 200, which is manufactured in such a way as to coat the blower fan with a photocatalyst, absorbs the ultraviolet rays radiated from the optical source unit 300, the electrons charged in a valence band (VB) absorb light energy and thus jump into a conduction band (CB) which is not filled with electrons. Consequently, holes, which are the sites in the valence band from which the electrons escape, oxidize water molecules on the surface thereof while returning to the original state thereof, and the oxidized water molecules generate hydroxyl radicals. The generated hydroxyl radicals decompose and eliminate the contaminants contained in the internal air or external air, introduced into the air conditioner case 100.

The photocatalytic unit 200 may include a plurality of optical source units 300 to generate hydroxyl radicals using the ultraviolet rays radiated from the inside and the entire external surface thereof.

The optical source unit 300 radiates ultraviolet rays to the photocatalytic unit 200 to cause a photocatalytic reaction in the photocatalytic unit 200, generating hydroxyl radicals for decomposing contaminants.

As illustrated in FIG. 4, the optical source unit 300 may include a first optical source 310 mounted inside the photocatalytic unit 200 and a second optical source 320 mounted outside the photocatalytic unit 200.

As illustrated in FIG. 5, the first optical source 310 includes a plurality of first optical sources mounted on a coupling mount 300a, which are provided at the center portion of the photocatalytic unit 200 and are connected to a printed circuit board PCB to radiate ultraviolet rays toward the internal surface of the photocatalytic unit 200.

Accordingly, the first optical source 310 radiates ultraviolet rays to contaminants for direct decomposition of the contaminants.

The plurality of first optical sources 310 may be mounted on the external circumferential surface of the coupling mount 300a, which is disposed at the center portion 200a of the cylindrical photocatalytic unit 200, at angular intervals of 90 degrees. In other words, four first optical sources 310 may be mounted on the external circumferential surface of the coupling mount 300a at the same angular interval.

Each of the first optical sources 310 is mounted at a predetermined height to face the internal surface of the photocatalytic unit 200 and to be inclined downwards.

In other words, the four first optical sources 310 are mounted on the external circumferential surface of the coupling mount 300a at an angular intervals of 90 degrees and are inclined downwards at a predetermined angle to obliquely radiate ultraviolet rays such that the radiated ultraviolet rays reach the entire internal surface of the photocatalytic unit 200 without omission of any area to be radiated. Consequently, the photocatalytic reaction efficiently occurs in the photocatalytic unit 200.

The second optical source includes the same number of second optical sources as the number of the first optical sources 310 mounted in the air conditioner case 100 to radiate ultraviolet rays to the external surface of the photocatalytic unit 200.

The number of second optical sources 320 is four, which is the same as the number of first optical sources 310. The four second optical sources 320 are radially disposed around the center portion of the photocatalytic unit 200 to respectively face the first optical sources 310 (see FIG. 4).

The second optical sources 320 may be configured not only to radiate ultraviolet rays to the external surface of the photocatalytic unit 200 to thus cause photocatalytic reaction to efficiently occur at the photocatalytic unit 200 but also to directly radiate ultraviolet rays to contaminants to thus directly decomposed the same, as in the first optical sources 310.

As illustrated in FIG. 7, each of the second optical sources 320 is preferably mounted at a position on the air conditioner case 100 which is aligned with a line that extends through the center portion 200a of the photocatalytic unit 200.

If each of the second optical sources 320 is mounted at a position on the air conditioner case 100 that deviates in the rotation direction of the photocatalytic unit 200 from the position on the air conditioner case 100 which is aligned with a line that extends through the center portion 200a of the photocatalytic unit 200, as illustrated in FIG. 8, there is a loss area A of ultraviolet rays corresponding in size to the extent of deviation, compared to the case in which each of the second optical sources 320 is mounted at the position on the air conditioner case 100.

Therefore, a photocatalytic reaction cannot efficiently occur in the loss area A of the photocatalytic unit 200, with the result that the generation of hydroxyl radicals in the external region of the photocatalytic unit 200 is reduced, deteriorating the ability to degrade contaminants.

Accordingly, each of the second optical sources 320 be disposed to face a corresponding one of the first optical sources 310 and to be mounted at a position on the air conditioner case 100 which is aligned with a line that extends through the center portion 200a of the photocatalytic unit 200 to eliminate the loss area A.

As illustrated in FIG. 9, one of the second optical sources 320 may be selectively disposed with an optical lens 322, which is coupled thereto when the second optical source 320 is positioned at a distance from the external surface of the photocatalytic unit 200 which is shorter than a predetermined distance.

In other words, when one of the second optical sources 320 is positioned at a distance from the photocatalytic unit 200 which is shorter than a predetermined distance such that the second optical source 320 irradiates only a relatively small area of the external surface of the photocatalytic unit 200, the optical lens 322 may be selectively coupled to the second optical source 320 to increase the radiation angle of the second optical source 320.

When one 320 of the second sources is positioned at a shorter distance from the external surface of the photocatalytic unit 200 than is another 320′ of the second optical sources, as illustrated in FIG. 10, that is, at a distance shorter than a predetermined distance, the ultraviolet rays radiated from the one 320 of the second sources reaches only a relatively small area of the photocatalytic unit 200, whereby a photocatalytic reaction cannot efficiently occur in the remaining area of the photocatalytic unit 200.

Accordingly, the one 320 of the second optical sources which is positioned at a smaller distance from the photocatalytic unit 200 than the another 320′ of the second optical sources, is provided with the optical lens 322 to increase the radiation angle of ultraviolet rays such that the ultraviolet rays cover the entire outward-facing surface of the photocatalytic unit 200. As a result, since the photocatalytic reaction occurs on the entire external surface of photocatalytic unit 200, it is possible to improve the ability to eliminate contaminants.

As is apparent from the above description, the air sterilization device for a vehicular air conditioner according to various exemplary embodiments of the present invention includes the photocatalytic unit, which is manufactured in such a way as to coat a blower fan with a photocatalyst, and the optical sources configured to radiate ultraviolet rays to the photocatalytic unit to thus activate a photocatalytic reaction, generating hydroxyl radicals. The photocatalytic unit and the optical sources are mounted in an air path, into which internal air or external air is introduced, to perform sterilization using ultraviolet rays and sterilization resulting from the oxidizing action of the hydroxyl radicals generated by the ultraviolet rays, making it possible to efficiently degrade contaminants.

Furthermore, the plurality of optical source units are disposed outside the photocatalytic unit such that the optical source units are aligned with lines that extend through the center portion of the blower fan and such that each of the optical source units is selectively provided with an optical lens depending on the distance from the photocatalytic unit, with the result that there are effects of minimizing the loss area of the blower fan that ultraviolet rays cannot reach and thus of improving the efficiency with which contaminants are removed.

For convenience in explanation and accurate definition in the appended claims, the terms “upper”, “lower”, “inner”, “outer”, “up”, “down”, “upwards”, “downwards”, “front”, “rear”, “back”, “inside”, “outside”, “inwardly”, “outwardly”, “interior”, “exterior”, “internal”, “external”, “forwards”, and “backwards” are used to describe features of the exemplary embodiments with reference to the positions of such features as displayed in the figures. It will be further understood that the term “connect” or its derivatives refer both to direct and indirect connection.

The foregoing descriptions of specific exemplary embodiments of the present invention have been presented for purposes of illustration and description. They are not intended to be exhaustive or to limit the present invention to the precise forms disclosed, and obviously many modifications and variations are possible in light of the above teachings. The exemplary embodiments were chosen and described to explain certain principles of the present invention and their practical application, to enable others skilled in the art to make and utilize various exemplary embodiments of the present invention, as well as various alternatives and modifications thereof. It is intended that the scope of the present invention be defined by the Claims appended hereto and their equivalents.

What is claimed is:

1. An air sterilization device for a vehicular air conditioner, the air sterilization device comprising:
an air conditioner case, which includes an air inlet provided at an intake side of the air conditioner case and an air outlet provided at a discharge side of the air conditioner case and which includes an air path in the air conditioner case;
a photocatalytic unit, which is configured to blow internal air or external air toward the air outlet of the air conditioner case and a surface of which is coated with a photocatalyst; and
an optical source unit configured to radiate ultraviolet rays to the photocatalytic unit to thus cause a photocatalytic reaction at the photocatalytic unit, generating hydroxyl radicals.

2. The air sterilization device of claim 1, wherein the optical source unit includes:
a first optical source mounted on a coupling mount provided at a center portion of the photocatalytic unit to radiate ultraviolet rays to an internal surface of the photocatalytic unit; and
a second optical source configured to radiate ultraviolet rays to an external surface of the photocatalytic unit.

3. The air sterilization device of claim 2, wherein the first optical source includes a plurality of first optical sources, which are disposed on an external circumferential surface of the coupling mount positioned at the center portion of the photocatalytic unit at an angular intervals of 90 degrees and which are oriented to be inclined downwards and to face the internal surface of the photocatalytic unit.

4. The air sterilization device of claim 2, wherein the second optical source includes a plurality of second optical sources, a number of which is a same as a number of the plurality of first optical sources and which are mounted on the air conditioner case to face respective ones of the plurality of first optical sources.

5. The air sterilization device of claim 4, wherein the plurality of second optical sources is mounted at a position on the air conditioner case which is aligned with a line that extends through the center portion of the photocatalytic unit.

6. The air sterilization device of claim 2, wherein the second optical source selectively includes an optical lens coupled thereto when the second optical source is positioned at a distance from the external surface of the photocatalytic unit which is smaller than a predetermined distance.

7. The air sterilization device of claim 2, wherein one of the second optical sources which is positioned at a smaller distance from the photocatalytic unit than another of the second optical sources, is provided with an optical lens having a wider radiation angle of ultraviolet rays than the another of the second optical sources.

8. The air sterilization device of claim 1, wherein the photocatalytic unit includes a blower fan.
